(19) United States
(12) Patent Application Publication
(10) Pub. No.: US 2019/0348168 A1
(43) Pub. Date: Nov. 14, 2019

(54) DIAGNOSIS AND TREATMENT OPTIMIZATION FOR PATIENT DISORDERS

(71) Applicant: Opya, Inc., San Mateo, CA (US)

(72) Inventors: Jessica Hammond Owens, San Francisco, CA (US); Afton Kerry Vechery, San Francisco, CA (US)

(21) Appl. No.: 16/409,721

(22) Filed: May 10, 2019

Related U.S. Application Data
(60) Provisional application No. 62/669,725, filed on May 10, 2018.
(60) Provisional application No. 62/669,748, filed on May 10, 2018.

(51) Int. Cl. G16H 20/70; G16H 10/20; G09B 19/00; A61B 5/00

(52) U.S. Cl. CPC G16H 20/70; G16H 10/20; A61B 5/168; A61B 5/7475; G09B 19/00

(57) ABSTRACT

Method for managing treatment for a disorder of a patient that employs instructions to generate an interview dataset and a behavior detection dataset associated with a patient. The interview dataset is employed to provide tasks for the patient to perform while monitoring the patient for performance of different types of behaviors in the behavior detection dataset. The behavior detection dataset is transformed into an observation dataset based upon detection of monitored behaviors performed by the patient that corresponds to the behavior detection dataset. A characterization of the disorder of the patient is based upon the interview dataset and the observation dataset.

### CROSS-REFERENCE TO RELATED APPLICATIONS

This application is a Utility Patent application based on previously filed U.S. Provisional Patent Application Ser. No. 62/669,725 filed on May 10, 2018, and U.S. Provisional Patent Application Ser. No. 62/669,748 filed on May 10, 2018, the benefit of the filing date of which is hereby claimed under 35 U.S.C. § 119(e) and the contents of which are each further incorporated in entirety by reference.

### TECHNICAL FIELD

This invention relates generally to the neurology field, and more specifically to a new and useful method for characterizing disorders.

### BACKGROUND

Neuropsychiatry is a specialty of medicine crossing neurology and psychology, which entails mental disorders attributable to diseases of the nervous system. While many neuropsychiatric and neurological disorders are treatable, success of a treatment regimen relies heavily upon early diagnosis, identification of symptoms during key periods of development, accurate diagnosis, and formation of personalized therapies that match the patient and the diagnosis.

Unfortunately, current standards of diagnosis and treatment are responsible for delays in diagnoses of disorders and/or misdiagnoses of disorders, which cause the disorders to remain untreated or undertreated. While the delays are partially due to the non-intuitive, time-sensitive, and patient demographic-sensitive nature of such disorders, current standards of diagnosis are unnecessarily deficient in many aspects. Also, the current standards of diagnosis can be difficult to administer due to inherent differences between a diagnosis environment and a patient's natural environment. Additionally, these inherent deficiencies, further limitations in diagnosis, treatment, and/or monitoring of patient progress during treatment prevent adequate care of patients with diagnosable and treatable disorders.

Machine learning is increasingly playing a larger and more important role in developing and improving the understanding of complex patient disorders. As machine learning techniques have matured, machine learning has rapidly moved from the theoretical to the practical. Combined with the advent of big-data technology, machine learning solutions are being applied to a variety of industries and applications that until now were difficult, if not impossible to effectively reason about. As such, there has been a need for the development of different types of machine learning models that may be used for diagnosis and predicting treatment outcomes for different patient disorders.

At the same time, security regulations, often required by law, have made implementing, deploying, and servicing machine learning applications cumbersome. For example, the Health Insurance Portability and Accountability Act of 1996 (HIPAA) limits the use and disclosure of individually identifiable health information, which poses a technological challenge to machine learning applications that consume health information. For instance, individually identifiable health information may not be transferred outside of a secure environment, such as to a third party server, making it difficult to train models with or to apply models to protected data. Similarly, when a machine learning based application has an error exposed by a particular set of data, diagnosing or even reproducing the error can be difficult. As such, there is a need in the neurology field for a new and useful machine learning based facility for diagnosis and treatment of patient disorders.

### BRIEF DESCRIPTION OF THE DRAWINGS

Non-limiting and non-exhaustive embodiments of the present innovations are described with reference to the following drawings. In the drawings, like reference numerals refer to like parts throughout the various figures unless otherwise specified. For a better understanding of the described innovations, reference will be made to the following Detailed Description of the Various Embodiments, which is to be read in association with the accompanying drawings, wherein:

FIG. 1 illustrates a flow chart for a method to characterize disorders;

FIG. 2 shows a schematic embodiment of a portion of a method for characterizing disorders;

FIGS. 3A and 3B illustrate different graphs showing the method for characterizing disorders; and

FIG. 4 shows a logical schematic of a system for characterizing disorders in accordance with the invention.

### DESCRIPTION OF VARIOUS EMBODIMENTS

Various embodiments now will be described more fully hereinafter with reference to the accompanying drawings, which form a part hereof, and which show, by way of illustration, specific exemplary embodiments by which the invention may be practiced. The embodiments may, however, be embodied in many different forms and should not be construed as limited to the embodiments set forth herein; rather, these embodiments are provided so that this disclosure will be thorough and complete, and will fully convey the scope of the embodiments to those skilled in the art. Among other things, the various embodiments may be methods, systems, media or devices. Accordingly, the various embodiments may take the form of an entirely hardware embodiment, an entirely software embodiment or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. Also, throughout the specification and the claims, the use of “when” and “responsive to” do not imply that associated resultant actions are required to occur immediately or within a particular time period. Instead they are used herein to indicate actions that may occur or be performed in response to one or more conditions being met, unless the context clearly dictates otherwise. In addition, throughout the specification, the meaning of “a,” “an,” and “the” include plural references. The meaning of “in” includes “in” and “on.”

The following briefly describes the various embodiments to provide a basic understanding of some aspects of the invention. This brief description is not intended as an extensive overview. It is not intended to identify key or critical elements, or to delineate or otherwise narrow the scope. Its purpose is merely to present some concepts in a simplified form as a prelude to the more detailed description that is presented later.

Briefly stated, embodiments are directed towards managing diagnosis and treatment of patient disorders. A method, system, or processor readable non-transitory storage media for managing treatment for a disorder of a patient, comprising: (1) employing a set of instructions to generate one or more of an interview dataset and a behavior detection dataset that includes a set of different types of behaviors associated with a patient; (2) employing the interview dataset to provide one or more tasks for the patient to perform while monitoring the patient for performance of the different types of behaviors in the behavior detection dataset; (3) transforming the behavior detection dataset into an observation dataset based upon detection of one or more monitored behaviors performed by the patient that corresponds to the behavior detection dataset; (4) generating a characterization of the disorder of the patient based upon the interview dataset and the observation dataset; (5)providing one or more goals for one or more therapy regimens for the patient based at least upon the characterization of the patient's disorder; (6) employing participation by the patient in the one or more therapy regimens over time to generate a set of values for one or more metrics associated with the disorder; and (7) modifying the one or more therapy regimens based on based on the set of values of the one or more metrics and progress towards meeting the one or more goals.

In one or more embodiments, providing a task to the patient at a user interface for an application on an electronic device. The task is configured to prompt one or more of the set of different types of behaviors by the patient. Employing the user interface to automatically detect performance of the one or more different types of behaviors by the patient.

In one or more embodiments, transforming the behavior detection dataset into the observation dataset further comprises employing video data over time to detect the at least one behavior performed by the patient that corresponds to the set of different types of behaviors.

In one or more embodiments, transforming the behavior detection dataset into the observation dataset further comprises employing audio data over time to detect the at least one behavior performed by the patient that corresponds to the set of different types of behaviors.

In one or more embodiments, transforming the behavior detection dataset into the observation dataset further comprises employing one or more sensor devices to detect the at least one behaviors performed by the patient that corresponds to the set of different types of behaviors.

In one or more embodiments, the provided one or more tasks further comprise one or more of: (1) a first task to prompt a neuro-typical behavior by the patent; (2) a second task to prompt a neuro-atypical behavior by the patient; or (3) a third task to prompt emotional significance by the patient.

In one more embodiments, modifying the characterization of the disorder of the patient based on the set of values of the one or more metrics and progress towards meeting the one or more goals.

In one or more embodiments, additional actions can include: (a) providing a task to the patient at a user interface, the task being configured to prompt a behavior listed in the set of different types of behaviors; and automatically detecting performance of the behavior by the patient, based upon at least one interaction between the patient and one or more of the user interface or a biometric component.

The one or more embodiments provide for accurate and efficient diagnosis of a neurological disorder and generation of a treatment plan for a patient. Also, a disorder, such as a neurological disorder, may be detected at an appropriate stage such that any prescribed therapy regimens are more likely to have a high level of effectiveness. Further, additional therapy regimens can be recommended for inclusion in the treatment plan based upon characterization of the disorder, as well as monitoring actions of the subject during participation in a therapy regiment (e.g., in an automated manner with biometric devices) to provide for more personalization of the treatment plan for the patent with a disorder.

In one or more embodiments, the patient's disorder can include any one or more of: autism, attention deficit disorder, oppositional defiant disorder, specific learning disorders, a speech disorder/impairment, attention deficit hyperactive disorder, Tourette's Syndrome, obsessive compulsive disorder, sensory integration disorder, depression, and any other neurological disorder.

One of the benefits of the various embodiments is accurately, effectively, and timely providing a diagnosis of autism in a patient, which is currently hindered by delays (e.g., bureaucratic delays) in connecting potential autism patients with practitioners able to provide a diagnosis. In some instances, such delays can approach 9-12 months, during which further progression of the disorder may have occurred, and during which a key treatment opportunity may have passed. In characterizing an autism diagnosis for a patient, the characterization can include a diagnosis of severity and a type (e.g., Classical Autism, Asperger's Syndrome, Childhood Disintegrative Syndrome, Rett's Disorder, Pervasive Developmental Disorder—Not Otherwise Specified, etc.) within a spectrum of autism disorders. Furthermore, the diagnosis or characterization of autism can be performed for subjects of any suitable demographic (e.g., age demographic, ethnicity, gender, socioeconomic demographic, health condition, etc.). For example, the various embodiments can diagnose severity and type of autism spectrum disorder for child or adolescent subjects exhibiting symptoms of autism, to provide treatment recommendations to such patients, and to monitor such patients during treatment of their disorder(s). Additionally, in one or more of the embodiments, any other suitable neurological or non-neurological disorder can be characterized or diagnosed, for any other suitable demographic of patients.

### Generalized Operations

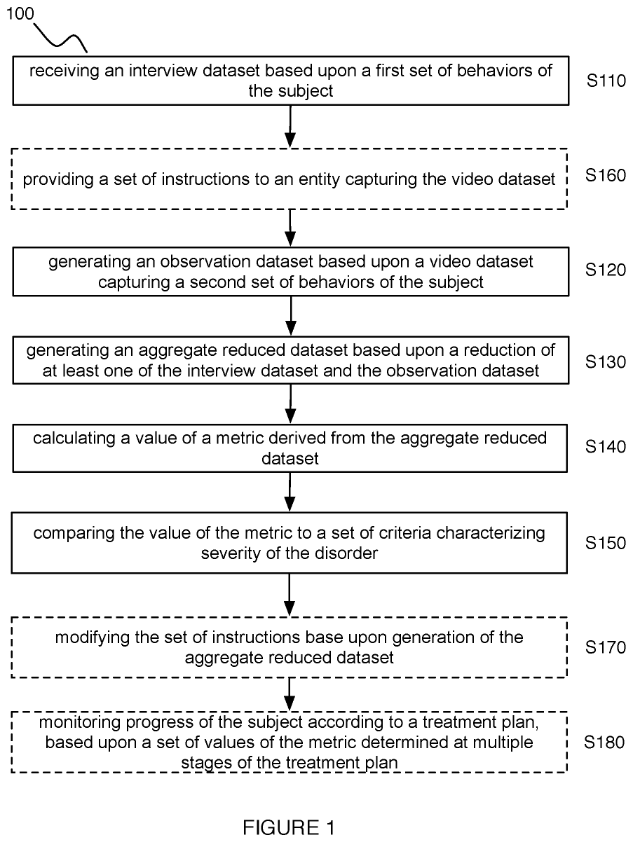

As shown in FIG. 1, an embodiment of a method 100 for determining a state of a disorder potentially characterizing a subject comprises: receiving an interview dataset based upon a first set of behaviors of the subject S110; generating an observation dataset based upon a video dataset capturing a second set of behaviors of the subject S120; generating an aggregate reduced dataset based upon a reduction of at least one of the interview dataset and the observation dataset S130; calculating a value of a metric derived from the aggregate reduced dataset S140; and comparing the value of the metric to a set of criteria characterizing the disorder S150, thereby characterizing the state of the disorder for the subject based upon the metric. The method 100 can additionally or alternatively include one or more of: providing a set of instructions to an entity capturing the video dataset S160, wherein the set of instructions guides the entity in documenting elements of the observation dataset; modifying the set of instructions based upon the aggregate reduced dataset S170, thereby increasing efficiency in capturing a subsequent video dataset; and monitoring progress of the subject according to a treatment plan, based upon a set of values of the metric determined at multiple stages of the treatment plan S180.

The method 100 functions to enable a disorder of a subject to be accurately characterized, and preferably, enables a neurological disorder of a subject to be accurately characterized. As such, the method 100 can aid in the characterization, diagnosis, and/or treatment of a disorder, and furthermore, to enable detection of the disorder at an appropriate stage such that any prescribed treatments can have a high level of effectiveness.

In some applications of the method 100, the disorder can include any one or more of: autism, attention deficit disorder, oppositional defiant disorder, specific learning disorders, a speech disorder/impairment, attention deficit hyperactive disorder, Tourette's Syndrome, obsessive compulsive disorder, sensory integration disorder, depression, and any other neurological disorder. In some variations, the method 100 can facilitate diagnosis of autism in a subject, which is currently hindered by delays (e.g., bureaucratic delays) in connecting potential autism patients with practitioners able to provide a diagnosis. In some instances, such delays can approach 9 to 12 months, during which progression of the disorder has occurred, and during which a key treatment opportunity has passed. In these variations for characterizing autism in a subject, the diagnosis can include a diagnosis of severity and further characterization of type (e.g., Classical Autism, Asperger's Syndrome, Childhood Disintegrative Syndrome, Rett's Disorder, Pervasive Developmental Disorder—Not Otherwise Specified, etc.) within the spectrum of autism disorders. Furthermore, the diagnosis or characterization can be performed for subjects of any suitable demographic (e.g., age demographic, ethnicity, gender, socioeconomic demographic, health condition, etc.). In a specific example of these variations, the method 100 can be used to diagnose severity and type of autism spectrum disorder for child or adolescent subjects exhibiting symptoms of autism. However, in other variations of the method 100, any other suitable neurological or non-neurological disorder can be characterized or diagnosed, for any other suitable demographic of subjects.

Block S110 recites: receiving an interview dataset based upon a first set of behaviors of the subject, and functions to receive a survey of information reported regarding the subject that can be used to characterize the state of the disorder for the subject. Preferably, the interview dataset is generated based upon responses to a set of items of a survey, wherein the responses are provided by an entity who has directly or indirectly observed the first set of behaviors of the subject; however, the interview dataset can additionally or alternatively be generated in any other suitable manner. In variations, the entity can be any one or more of: a parent, a sibling, a healthcare provider, a supervisor, a peer, and any other suitable entity able to accurately provide responses to the set of items of the survey. Furthermore, the survey is preferably provided to the entity electronically (e.g., at a mobile application, at a web application, using a messaging client, using an email client, etc.); however, the survey can additionally or alternatively be provided to the entity non-electronically (e.g., by paper, verbally, etc.).

In one or more embodiments, the survey can be provided to the entity in modules, wherein the modules are provided upon one or more triggers (e.g., a behavior of the subject can trigger provision of a module of the survey), at regular or irregular intervals of time (e.g., at certain ages or developmental stages of the subject), and/or in any other suitable manner. Additionally or alternatively, the survey can be provided to the entity in completion. In examples of Block S110 for characterization of autism in a subject, the interview dataset can be derived from a survey comprising content of the Autism Diagnostic Interview-Revised (ADI-R). As such, the interview dataset can include responses to 93 items (or any other suitable number of items) of the ADI-R survey divided into three behavioral areas including 1) social interaction, 2) communication and language, and 3) restricted and repetitive behaviors.

However, in other examples of Block S110 for autism characterization, the interview dataset can additionally or alternatively include responses to a survey comprising content derived from any one or more of: the Autism Diagnostic Interview (ADI), the Social Communication Questionnaire (SCQ), and any other suitable instrument configured to facilitate documentation of behaviors indicative or not indicative of autism. Alternatively, the interview dataset can additionally or alternatively be derived from any other suitable instrument, survey, and/or diagnostic manual (e.g., a version of the Diagnostic and Statistical Manual of Mental Disorders) configured to characterize a state of any other suitable disorder.

In variations of Block S110 for characterization of an autism state in a subject, the first set of behaviors preferably includes behaviors related to any one or more of: communication and language skills (e.g., speech development, appropriate word use, ability to sustain a conversation, etc.), social interaction issues (e.g., emotional response interpretation, display of emotional responses, irregularities in focus, irregularities in making eye contact, etc.), repetitive and obsessive behaviors (e.g., fixation on items, repetition of words or phrases out of context, repetitive motions such as flapping or pacing, etc.), ability to perform tasks (e.g., pointing, showing) when prompted, and any other suitable behavior indicative or not indicative of a state of autism. The first set of behaviors can include behaviors exhibited or not exhibited currently by the subject, and can additionally or alternatively include behaviors exhibited or not exhibited by the subject at a past time point (e.g., when the subject was at a given age or within a range of ages prior to the present). In these variations for autism, the first set of behaviors is preferably observed and captured in the interview dataset for a subject greater than 18 months of age; however, the first set of behaviors can additionally or alternatively be determined for a subject of any suitable age demographic. As such, responses to the survey provided in variations of Block S110, contributing to the interview dataset, can identify whether the subject exhibits behaviors indicative or not indicative of a state of autism, based upon observation of the first set of behaviors by an overseeing entity. In variations of the method for characterizing non-autism disorders, Block S110 can include receiving any other suitable dataset based upon any other suitable set of behaviors or factors (e.g., biometric, genetic, etc.) of the subject.

The interview dataset preferably includes a quantified score for the response(s) to each item and/or group of items of a survey, which can be processed and/or reduced to determine a metric according to Blocks S130 and S140. The quantified score(s) for each item or group of items can be generated from a set of qualitative criteria, wherein each qualitative criterion of the set of qualitative criteria is mapped to a quantified metric (e.g., a number along a scale). However, the quantified score(s) for each item or group of items can additionally or alternatively be generated in any other suitable manner. For instance, the number of instances in which a subject exhibits a behavior (e.g., total number, number within a given time period, difference in number between different time points or time periods, etc.) can be used to generate a quantified score for an item of the survey. In a specific example case for autism characterization according to the ADI-R, responses to each of 93 items (or any other suitable number of items) can be scored on a scale from zero to nine, wherein a score of zero indicates that a “behavior of the type specified in the coding is not present”, a score of one indicates that a “behavior of the type specified is present in an abnormal form, but not sufficiently severe or frequent to meet the criteria for a 2”, a score of 2 indicates “definite abnormal behavior”, a score of 3 indicates “extreme severity of the specified behavior”, a score of 7 indicates “definite abnormality in the general area of the coding, but not of the type specified”, a score of 8 indicates “not applicable”, and a score of 9 indicates “not known or asked”.

With regard to the ADI-R, scores from individual items are aggregated to generate scores for each of three behavioral areas (e.g., by one or more of averaging, adding, weighting, and subtracting scores), which can be used to determine a metric in variations of Blocks S130 and S140. Variations of the specific example can, however, include generation and/or aggregation of quantified scores from the first set of behaviors, in any other suitable manner. Alternatively, generation of the interview dataset may not include generation of one or more quantified scores.

Block S120 recites: generating an observation dataset based upon a video dataset capturing a second set of behaviors of the subject, and functions to receive an additional set of information regarding the subject that can be used to characterize the state of the disorder for the subject. In variations of Block S120 for characterization of an autism state, the observation dataset and/or the video dataset are preferably generated according to methods derived from the Autism Diagnostic Observation Schedule (ADOS) or a variation thereof (e.g., ADOS-2, ADOS-G, etc.) and can additionally or alternatively include annotated methods of the ADOS and/or items not included in the ADOS. Alternatively, the observation dataset and/or the video dataset can be generated according to any other suitable instrument for characterization of autism or any other suitable disorder based upon observation of behaviors. The video dataset is preferably captured in real time; however, the video dataset can alternatively be captured in non-real time. Furthermore, the video dataset preferably includes a set of video clips, taken at different time points; however, the video dataset can alternatively be a continuous video stream spanning a duration of time without any breaks. In variations wherein the video dataset includes a set of video clips, each video clip in the set of video clips can span any suitable duration of time and/or can be received in real or non-real time. Furthermore, capture of each video clip can be triggered automatically (e.g., based upon sensor detection of a behavior) or performed manually.

Preferably, the video dataset is generated by guiding an entity in communication with the subject to capture the video dataset, wherein the entity can be the same entity as in variations of Block S110, or a different entity. In variations, the entity can be any one or more of: a parent, a sibling, a healthcare provider, a supervisor, a peer, and any other suitable entity able to accurately provide responses to the set of items of the survey. Furthermore, guidance of the entity in capturing the video dataset is preferably performed by providing the entity with a set of instructions electronically (e.g., at a mobile application, at a web application, using a messaging client, using an email client, using audio, using video, etc.) at a user interface of a device able to capture the video dataset; however, the set of instructions can additionally or alternatively be provided to the entity non-electronically or electronically (e.g., by paper, verbally, visually, etc.) at an interface separate from that of a device able to capture the video dataset. The instructions preferably guide the entity in administering tasks or activities to the subject, in order to prompt at least one behavior, but can additionally or alternatively guide the entity in passively capturing behaviors of the subject. However, the video dataset can additionally or alternatively be generated in any other suitable manner (e.g., based upon automatic capture, based upon capture by a non-human entity, etc.).

In variations of Block S120 for characterization of an autism state in a subject, the second set of behaviors preferably includes behaviors related to any one or more of: behaviors prior to and post development of motor coordination skills (e.g., walking), behaviors prior to competency in using phrase speech, behaviors post usage of phrase speech but prior to language fluency, behaviors post language fluency, pre-adolescent behaviors, and post-adolescent behaviors. Similar to Block S110, the second set of behaviors can additionally or alternatively include behaviors related to one or more of: communication and language skills (e.g., speech development, appropriate word use, ability to sustain a conversation, etc.), social interaction issues (e.g., emotional response interpretation, display of emotional responses, irregularities in focus, irregularities in making eye contact, etc.), repetitive and obsessive behaviors (e.g., fixation on items, repetition of words or phrases out of context, repetitive motions such as flapping or pacing, etc.), ability to perform tasks (e.g., pointing, showing) when prompted, and any other suitable behavior indicative or not indicative of a state of autism. In these variations for autism, the second set of behaviors is preferably observed and captured in the video dataset for a subject who exhibits some motor coordination (e.g., walking); however, the second set of behaviors can additionally or alternatively be captured for a subject of any suitable age or developmental stage demographic.

The observation dataset is generated based upon the video dataset, and preferably includes documentation of the second set of behaviors captured in the video dataset. As such, generation of the observation dataset can include manual processing, semi-automatic processing, and/or automatic processing of the video dataset to extract behaviors of the second set of behaviors indicative of a disorder state or not indicative of a disorder state (e.g., according to the ADOS, according to the ADOS-2, according to any suitable instrument, etc.). Manual or semi-automatic processing of the video dataset can include identifying behaviors indicative of the disorder state or not indicative of the disorder state by an analyst (e.g., human analyst) examining the video dataset.

In one or more embodiments, semi-automatic or automatic processing of the video dataset can include automatic identification of behaviors indicative of the disorder state or not indicative of the disorder state by a processor analyzing the video dataset, wherein the processor implements a visual detection algorithm for identifying one or more behaviors. In some variations of semi-automatic or automatic processing, the visual detection algorithm can implement machine learning algorithms that improve detection of such behaviors based upon acquisition of additional data and/or implementation of a training dataset (e.g., a set of data including captured and identified behaviors to train the machine learning algorithms). The observation dataset can thus be generated in near-real time upon reception of the video dataset, or in non-real time. As such, transformation of the video dataset into an observation dataset helps identify whether the subject exhibits behaviors indicative or not indicative of a disorder state (e.g., a state of autism), based upon capture of the second set of behaviors in the video dataset. In variations of the method for characterizing non-autism disorders, Block S110 can include receiving any other suitable dataset based upon any other suitable set of behaviors or factors (e.g., biometric, genetic, etc.) of the subject.

The observation dataset preferably includes a quantified score for at least one behavior of the second set of behaviors captured in the video dataset, which can be processed and/or reduced to determine a metric according to Blocks S130 and S140. The quantified score(s) for the captured behavior(s) can be generated from a set of qualitative criteria, wherein each qualitative criterion of the set of qualitative criteria is mapped to a quantified metric (e.g., a number along a scale). However, the quantified score(s) for each item or group of items can additionally or alternatively be generated in any other suitable manner. For instance, the number of instances in which a subject exhibits a behavior (e.g., total number, number within a given time period, difference in number between different time points or time periods, etc.), as captured in the video dataset, can be used to generate a quantified score for the observed behavior. The quantified score(s) can be generated by the entity of either Block S110 and S120, a trained analyst, a processor, and/or any other suitable entity. In a specific example case for autism characterization according to the ADOS, scoring of behaviors according to modules targeted to different stages of development (e.g., motor skill development, speech development, etc.) can including mapping of an “intensity” of a behavior to a quantified score. Furthermore, with regard to the ADOS, quantified scores from each behavior and/or module can be aggregated to generate one or more aggregate scores, in order to determine a metric in variations of Blocks S130 and S140. Variations of the specific example can, however, include generation and/or aggregation of quantified scores from the second set of behaviors, in any other suitable manner. Alternatively, generation of the observation dataset may not include generation of one or more quantified scores.

In other variations of Block S110 and S120, any other suitable instruments for characterizing or diagnosing a disorder, or instruments derived from these instruments, can be implemented with respect to the subject to generate suitable datasets. Furthermore, the datasets can be overlapping, which allows for verification of behaviors reported or captured in different manners. As such, overlapping datasets can facilitate authentication (e.g., by redundancy) of a reported behavior, identification of contractions between reported and observed behaviors, and/or can be used for any other suitable purpose. For instance, an entity-reported behavior according to a survey may be verified by a behavior captured in a video dataset. Additionally or alternatively, at least some portions of multiple datasets can be complementary, in order to characterize a more complete set of behaviors exhibited by the subject. For example, some entity-reported behaviors may be difficult to capture in a video dataset, and some behaviors capturable in a video dataset may not be easily recognized or reported by an entity.

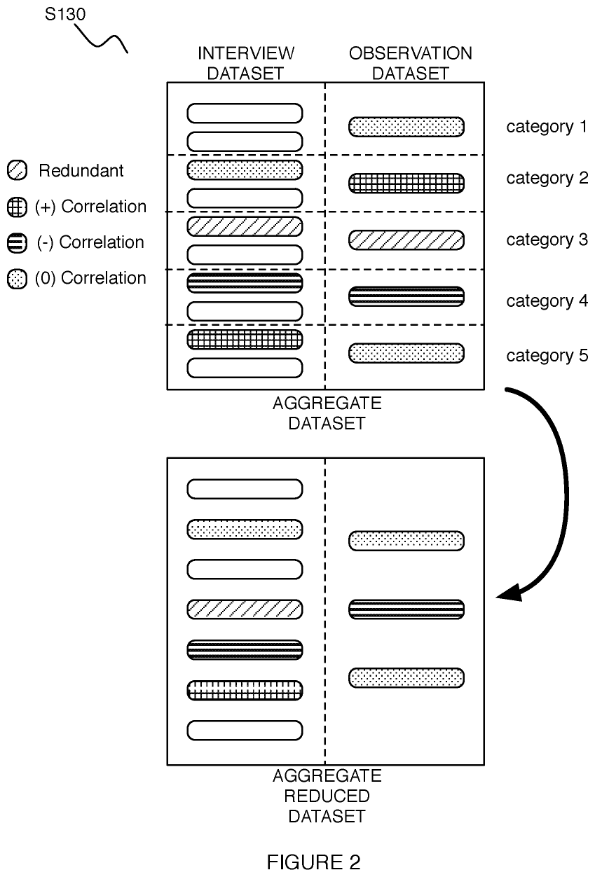

Block S130 recites: generating an aggregate reduced dataset based upon a reduction of at least one of the interview dataset and the observation dataset, and functions to reduce redundancy in and/or increase the efficiency of acquisition of the interview dataset and the observation dataset. Generating the aggregate reduced dataset can be performed prior to, subsequent to, or simultaneously with reduction of at least one of the interview dataset and the observation dataset. As shown in FIG. 2, a variation of a portion of the method for generating the aggregate reduced dataset is shown.

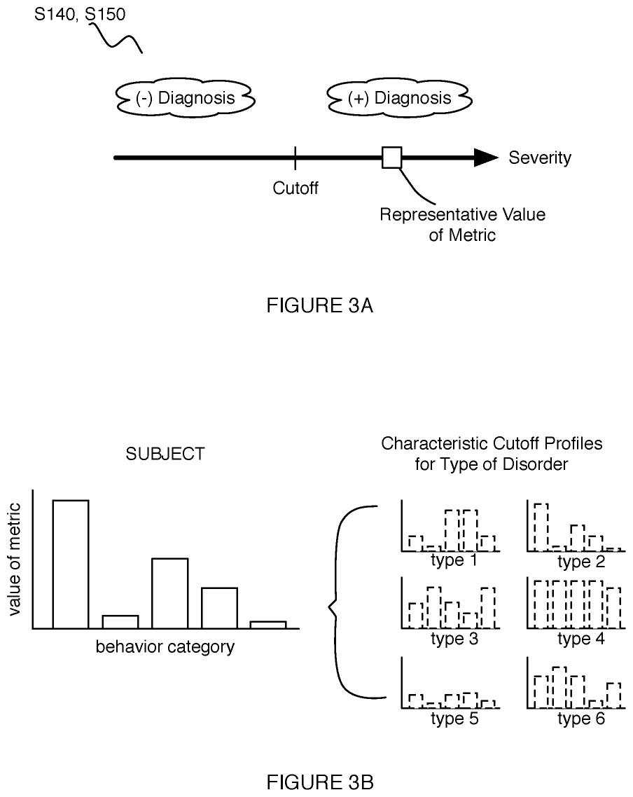

In a first variation, as shown in FIG. 3A, the interview dataset and the observation dataset can be aggregated prior to reduction, wherein aggregation includes grouping quantified scores of the interview dataset and the observation dataset by behavior category. In an example for autism characterization, all scores for behaviors of the first and the second set of behaviors related to social interaction can be grouped in a first category, all scores for behaviors of the first and the second set of behaviors related to communication and language can be grouped in a second category, and all scores for behaviors of the first and the second set of behaviors related to restricted and repetitive behaviors can be grouped in a third category, thus aggregating the interview and the observation datasets, and organizing the aggregate dataset into groups. However, in variations of the first variation, aggregation can be performed in any other suitable manner (e.g., with or without grouping).

After aggregation of the interview and the observation datasets in the first variation, the aggregate dataset can be reduced according to any suitable algorithm to account for redundancy, contradictions, and/or any other suitable artifact of the aggregate dataset. As such, reducing can include any one or more of: omitting scores based upon an identified redundancy, weighting scores based upon an identified redundancy (e.g., one or more scores for redundant items from the interview dataset and the observation dataset can be given a lower weight in generation of the aggregate reduced dataset), weighting scores based upon an identified importance (e.g., one or more scores for important items from the interview dataset and the observation dataset can be given a higher weight in generation of the aggregate reduced dataset), adding scores based upon an identified importance (e.g., one or more scores for important items from the interview dataset and the observation dataset can be added in generation of the aggregate reduced dataset), subtracting scores based upon an identified importance (e.g., scores from important items in the interview dataset and the observation dataset can be subtracted from each other in generation of the aggregate reduced dataset), averaging scores (e.g., determining a mean, a median, a mode) based upon an identified importance (e.g., multiple scores can be averaged in generation of the aggregate reduced dataset), and any other suitable mathematical operation that can be performed for scores from the aggregate dataset. In these variations, importance can be determined based upon a finding of efficacy or non-efficacy in accurately determining a state of the disorder, based upon data from the subject (e.g., from repeat datasets) and/or a group of subjects (e.g., of the same demographic as the subject, of a different demographic to the subject). Furthermore, scores from the aggregate dataset can be paired prior to reduction according to any other the above methods, whereby pairing can be performed based upon identification of a positive correlation between scores from each of the interview and the observation datasets, a negative correlation between scores from each of the interview and the observation datasets, or no correlation between scores from each of the interview and the observation datasets. One or more embodiments of the first variation include weighting, weighting can be performed using a measure of variance (e.g., standard deviation, correlation, variance, etc.) between items grouped according to behavioral category, paired according to correlation, grouped according to redundancy, or grouped by any other suitable means, in order to determine an appropriate weight as a measure of confidence. Then, a determined weight can be multiplied with the score(s) during reduction to form the aggregate reduced dataset. In these variations “higher weights” can be greater than zero or one, and “lower weights” can be less than one or zero. In examples of weighting, a positive correlation can be used to attribute a higher weight or a lower weight to one or more items that are positively correlated, a negative correlation can be used to attribute a higher weight, a lower weight, or a weight of zero to one or more items that are negatively correlated, a zero correlation can be used to attribute a lower weight or a weight of zero to non-correlated items, a lower weight or a weight of zero can be attributed to grouped items that have high variability, and a higher weight or a lower weight can be attributed to grouped items that have low variability. Reduction can thus be performed based upon analysis of the aggregate dataset, the interview dataset, and/or the observation dataset, and can additionally or alternatively be performed based upon historical data pertaining to demographics including, similar to, and/or different from the subject.

In a second variation of Block S130, reduction of the interview dataset and the observation dataset is performed prior to generation of the aggregate reduced dataset. The reduction of the interview dataset and the observation dataset in the second variation is performed based upon analysis of historical data for demographics including, similar to, and/or different from the subject. In one or more embodiments, the reduction is based upon data reduction techniques including one or more of: alternating decision tree analysis, best-first decision tree analysis, decision stump tree analysis, functional tree analysis, C4.5 decision tree analysis, repeated incremental pruning analysis, logistic alternating decision tree analysis, logistic model tree analysis, nearest neighbor generalized exemplar analysis, association analysis, divide-and-conquer analysis, random tree analysis, decision-regression tree analysis with reduced error pruning, ripple down rule analysis, classification and regression tree analysis, and any other suitable reduction analysis technique. In the second variation, the reduction is performed using the same reduction technique(s) for each of the interview dataset and the observation dataset separately prior to aggregation of the reduced datasets; however, the reduction can be performed using different techniques for each of the interview dataset and the observation dataset prior to aggregation.

In aggregating the reduced interview and observation datasets of the second variation, Block S130 can include grouping items of the reduced datasets based upon any one or more of: similarity in observed behavior, positive correlation in quantified score, negative correlation in quantified score, no correlation in quantified score, an identified importance, and any other suitable factors. Alternatively, aggregation can be performed without grouping, and/or can include any one or more of: adding scores, weighting scores (e.g., based upon importance, based upon a measure of variance), subtracting scores, averaging scores, omitting scores, and any other suitable mathematical operation as described in relation to the first variation above. Finally, in some variations of the second variation a secondary reduction can be performed to arrive at the aggregate reduced dataset, which can include any one or more of: omission, weighting, subtraction, adding, and averaging of scores for redundant, important, or non-important items.

In the described variations of Block S130, reducing at least one of the interview dataset and the observation dataset according to historical or non-historical data from the subject or demographic of subjects can include implementation of a machine learning algorithms, which can be trained based upon data from the subject and/or data from demographics including, similar to, and/or different from the subject. As such, by accumulation of data and machine learning, identification of items known to correlate with each other, known to compete with each other known to negate each other, known to be indicative, alone or in combination, of a disorder state, and/or known to have some importance in any other suitable manner can contribute to generation of the aggregate reduced dataset. Furthermore, aggregation and/or reduction can be performed according to any suitable combination of the above-described methods, and can be performed any suitable number of times and in any suitable order. Block S140 recites: calculating a value of a metric derived from the aggregate reduced dataset, and functions to derive at least one value of a metric for comparison to a set of criteria for determining a state of the disorder for the subject. Calculating the value of the metric can including any one or more of: averaging, adding, and weighting (e.g., based upon a measure of variance) all or a subset of the aggregate reduced dataset, with or without grouping based upon a common feature (e.g., behavior category).

In variations of Block S140 with grouping, calculating the value of the metric preferably includes calculating a value for each of a set of metrics, including at least one metric for each group (e.g., behavior category) characterized in the aggregate reduced dataset. Every value of a metric of the set of metrics is preferably determined in an identical manner using one or more of the above described techniques; however, one or more values of metrics of the set of metrics can alternatively be determined in a manner different from that of another value of a metric of the set of metrics. In relation to calculating a value of a metric for the aggregate reduced dataset, in some variations of Block S140 the value of the metric can be a value of a representative metric derived from the set of metrics determined for the set of groups. For instance, in some variations, all values for the set of metrics corresponding to behavior categories can be added together or averaged in order to determine a value of a representative metric. In one such example for autism characterization, with regard to the ADI-R and the ADOS, scores of the aggregate reduced dataset corresponding to different behavior categories (e.g., social interaction, communication and language, restricted and repetitive behaviors, etc.) can be averaged to calculate a value of a metric for each behavior category. Then, the average of the values of the metrics for the behavior categories can be determined as the representative value for the subject. Alternatively, however, calculating the value of the metric(s) can be performed in any other suitable manner.

Block S150 recites: comparing the value of the metric to a set of criteria characterizing the disorder, and functions to characterize the state of the disorder for the subject based upon the metric(s) calculated in Block S140. The set of criteria can include criteria related to a phenotypic expression of the disorder (e.g., as in a digital phenotype), positive diagnosis of the disorder, negative diagnosis of the disorder, severity of the disorder, type (e.g., subcategory) of the disorder, and any other suitable criterion for characterization of the disorder. As shown in FIG. 3B, each criterion in the set of criteria preferably corresponds to a value (e.g., a cutoff value, a threshold value) or a range of values against which the value of the metric is compared. In variations of Block S140 in which a value for each of a set of metrics is determined, each of the set of metrics can have a separate set of criteria against which a corresponding value of a metric is compared. In these variations, a positive diagnosis of the disorder can then be determined if a subset or all values of the metrics determined in Block S140 satisfy threshold conditions according to their respective set of criteria.

In an example of Block S150 for autism characterization, values for metrics in each of a set of behavior categories (e.g., social interaction, communication and language, restricted and repetitive behaviors, etc.) can be compared to cutoff values as criteria for each of the set of behavior categories, wherein values exceeding the cutoff values for each of the behavior categories indicates a positive diagnosis of autism. In a specific example for the ADI-R, the cutoff value in the social interaction category is 10, the cutoff value in the communication and language value is 8 (if verbal) or 7 (if nonverbal), and the cutoff value in the restricted and repetitive behaviors category is 3, wherein values of metrics for the subject exceeding all of the respective cutoff values indicates a positive diagnosis of autism. In another example, as shown in FIG. 3B, a representative value determined across metrics for each of a set of behavior categories can be compared to a set of criteria (e.g., a numeric scale), wherein location within the set of criteria indicates not only positive or negative diagnosis, but confidence in diagnosis and/or severity of the disorder for the subject (e.g., distance from a cutoff value in the set of criteria can indicate positive or negative diagnosis, as well as severity of the disorder). Furthermore, in this example, comparisons between subsets of individual values of metrics and their respective sets of criteria (e.g., a set of criteria for communication behavior, a set of criteria for social interaction behavior, a set of criteria for restricted and repetitive behaviors, etc.) can be used to determine a phenotypic characterization of autism (e.g., classic autism, Asperger's syndrome, etc.) that the subject has.

As shown in FIG. 1, the method 100 can additionally or alternatively include Block S160, which recites: providing a set of instructions to an entity capturing the video dataset. Block S160 functions to guide an entity in documenting elements of the observation dataset, in order to increase the efficiency of characterizing the disorder for the subject. As described in relation to Blocks S110 and S120 above, the entity can be any entity who is associated well enough with the subject to reliably capture and/or prompt behaviors of the subject. In variations, the entity can be any one or more of: a parent, a sibling, a healthcare provider, a supervisor, a peer, and any other suitable entity. The set of instructions preferably guide the entity in administering tasks or activities to the subject, in order to prompt at least one behavior (e.g., flapping, repetitive motion, pointing, showing, social interaction behavior, emotional response behavior, attention behavior, etc.) that can be used to characterize the disorder, but can additionally or alternatively guide the entity in passively capturing behaviors of the subject.

In Block S160, the set of instructions is preferably presented to the entity at an electronic device of the entity (e.g., by way of an application executing at a mobile device of the entity), wherein the electronic device may or may not include a video capture module configured to capture the video dataset. In one example, an instruction of the set of instructions, configured to guide the entity in performing a task with the subject to prompt an indicative behavior, is provided to the user by way of an application executing at a mobile device of the user, wherein the mobile device includes a video camera for capturing video data of the indicative behavior. In this example the set of instructions can thus guide the entity in capturing a set of video clips, each video clip potentially including at least one behavior of the second set of behaviors. However, in other variations, the set of instructions can additionally or alternatively be provided to the user in Block S160 in any other suitable manner (e.g., by paper, verbally, etc.). Furthermore, the set of instructions can include a set of commands to a non-human entity, such that capture of the video dataset is performed automatically by way of a controller issuing commands to a video capture module.

Also shown in FIG. 1, the method 100 can additionally or alternatively include Block S170, which recites: modifying the set of instructions based upon the aggregate reduced dataset. Block S170 functions to adapt the set of instructions based upon the reduction(s) performed in variations of Block S130, thereby increasing efficiency in capturing a subsequent video dataset. In variations of Block S130 including identification of redundancy among items of the interview dataset and the observation dataset, the set of instructions for capturing subsequent video datasets (e.g., for the subject, for another subject, for groups of subjects, etc.) can be modified to omit at least one instruction for capturing a behavior of the subject, based upon the identified redundancy. For example, if one or more items of the interview and/or the observation dataset are known to correlate consistently in some manner with a specific behavior captured in the video dataset, the set of instructions can be modified to omit instructions for prompting/capturing video of the specific behavior. In some variations, the order in which the set of instructions is provided can be modified, according to the aggregate reduced dataset, to provide a decision tree of instructions that guide the entity in efficiently capturing the video dataset. In one variation, the set of instructions can be ordered to place instructions that capture behaviors corresponding to highly influential items of the interview/video datasets (e.g., based upon a finding of importance in generating the aggregate reduced dataset) toward the beginning of the set of instructions, wherein exhibition of a specified number of these behaviors by the subject obviates a need for capturing more video data. In another variation, the set of instructions can be ordered in branches according to findings from generation of the aggregate reduced dataset, such that documentation of one behavior leads to instructions for capturing behaviors along one branch of the set of instructions, and obviates a need for capturing video data for behaviors along another branch of the set of instructions.

In some variations of Block S170 for characterization of autism, the set of instructions can be modified according to findings from generation of the aggregate reduced dataset, based upon characterization of subjects exhibiting a certain type or severity of autism, or based upon any other suitable demographic feature. In one variation, the set of instructions can be modified to provide tasks or activities more effective in prompting a behavior from a subject characterized by a certain type or severity of autism, and/or characterized by a specific demographic (e.g., stage of development, gender, etc.). In another variation, the set of instructions can be modified to facilitate implementation of capturing video of the subject, based upon the subject's type of autism, severity of autism, and/or demographic. In one such example, an instruction can be modified to guide an entity in hiding a video capture module from a subject having a certain type of autism and of a specific demographic, due to a finding that subjects of this type of autism and demographic do not exhibit a behavior in the presence of a video capture module. In such variations, expected responses by the subject, following implementation of the set of instructions, can further be used as validation in characterizing the state of the disorder for the subject. Modifying the set of instructions in Block S170 can, however, be performed in any other suitable manner, or according to any other suitable factor.

Also shown in FIG. 1, the method 100 can additionally or alternatively include Block S180, which recites: monitoring progress of the subject according to a treatment plan, based upon a set of values of the metric determined at multiple stages of the treatment plan. Block S180 functions to utilize values of the metric or set of metrics, spaced apart in time during the treatment plan, such that the subject's progress according to the treatment plan can be quantitatively and/or qualitatively assessed. The points in time can be regularly spaced across the duration of the treatment plan, can be irregularly spaced (e.g., according to key points, milestones, or adjustments during the treatment plan), and/or can be spaced according to any other suitable factor. In some variations, determining the value(s) of the metric(s) can be performed prior to and/or after key points (e.g., adjustments in therapy, transitions in developmental stages, events pertinent to the subject, etc.) or milestones in the treatment plan, in order to further determine progress relative to the key point(s) or milestone(s). In variations wherein a representative value is determined from the aggregate reduced dataset and compared to a set of criteria to determine a type and severity of the disorder, the representative value for the subject can be determined at multiple time points during the treatment plan to monitor changes in the severity and/or type of the disorder for the subject. In variations wherein a values for different metrics (e.g., metrics according to behavior category) are determined from the aggregate reduced dataset and compared to corresponding sets of criteria (e.g., for each of a behavior category) to characterize other aspects of the disorder (e.g., a type of autism), the values for the different metrics for the subject can be determined at multiple time points during the treatment plan to monitor changes in these other aspects of the disorder for the subject. In monitoring progression of the disorder in the subject, any one or more of the following can be used: a rate of change across multiple values of a metric, differences (e.g., absolute differences, percent differences, relative differences, etc.) between different values of a metric, a measure of variability (e.g., a variance, a standard deviation, etc.) in values of a metric, an average of multiple values of a metric, any parameter derived from one of the above parameters, and any other suitable parameter for monitoring the subject.

In some variations, Block S180 can further include modifying the treatment plan according to an analysis of progress according to Block S180. The analysis can include any one or more of: an analysis of an expected progress according to the treatment plan, a comparison between the expected progress and the actual progress of the subject, an analysis of non-responsiveness to the treatment plan, an analysis of a detrimental response to the treatment plan by the subject, an analysis of potential substitutions, subtractions, or additions to the treatment plan (e.g., alternative therapies, alternative medications, unrecommended therapies, unrecommended medications, etc.), and any other suitable analysis of a parameter indicative of progress. The analysis can then be used to modify the treatment plan, followed by subsequent monitoring of the subject as described above. Monitoring progress of the subject and/or modifying the treatment plan can, however, be performed in any other suitable manner.

The method 100 can additionally or alternatively include any other suitable blocks or steps configured to facilitate acquisition, reception, and/or processing of datasets, in order to determine a state of a disorder potentially characterizing a subject. For instance, in some variations the method 100 can include providing a second set of instructions configured to facilitate generation of the interview dataset, and modifying the second set of instructions based upon findings during generation of the aggregate reduced dataset, using techniques similar to or different from those described in relation to Blocks S160 and S170 above. In some variations the method 100 can additionally or alternatively include providing one or more of: the value(s) of the metric(s), analyses of progress according to the treatment plan, a notification, or any other suitable feedback to an entity associated with the subject, in order to facilitate proper care of the subject. Additionally, as a person skilled in the field of neurology will recognize from the previous detailed description and from the figures and claims, modifications and changes can be made to the embodiments, variations, examples, and specific applications of the method 100 described above without departing from the scope of the method 100.

### Illustrated Operating Environment

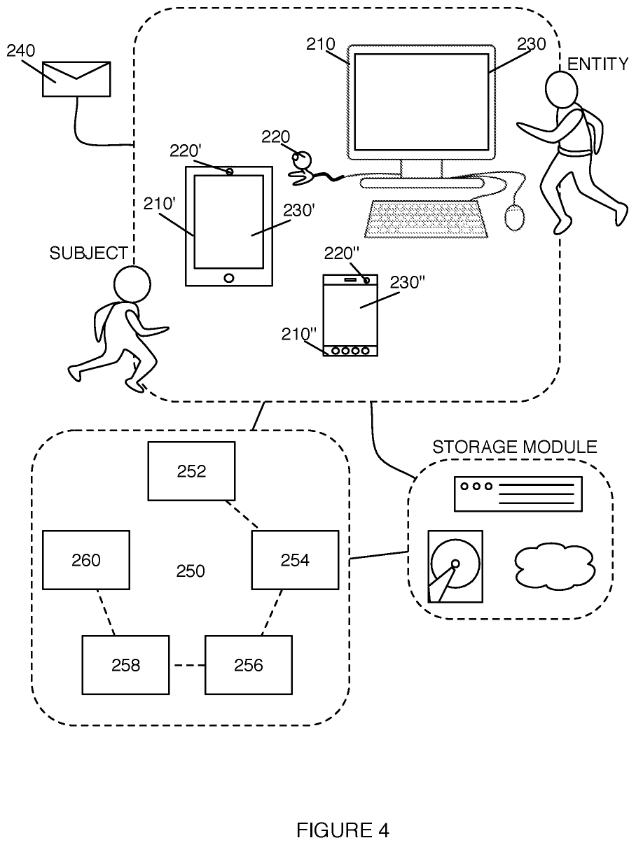

As shown in FIG. 4, a system is illustrated for determining a state of a disorder potentially characterizing a subject comprises a computing device 210 coupled to a video capture module 220, a user interface 230, and a message client 240; and a processor 250 comprising a first module 252 configured to receive an interview dataset based upon a first set of behaviors of the subject and a video dataset capturing a second set of behaviors of the subject from the video capture module 220, a second module 254 configured to generate an observation dataset based upon the video dataset, a third module 256 configured to generate an aggregate reduced dataset based upon a reduction of at least one of the interview dataset and the observation dataset, a fourth module 258 configured to calculating a value of a metric derived from the aggregate reduced dataset, and a sixth module 260 configured to compare the value of the metric to a set of criteria characterizing severity of the disorder, thereby characterizing the state of the disorder for the subject based upon the metric. The system is preferably configured to perform an embodiment of the method 100 described above, and can additionally or alternatively be configured to perform any other suitable method.

Computing device 210 may typically connect to other computing devices using a wired or wireless communications medium, e.g., personal computers, multiprocessor systems, microprocessor-based or programmable electronic devices, network PCs, or the like. In some embodiments, computing device 210 may include virtually any portable computer capable of connecting to another computer and receiving information such as, a laptop computer, a mobile computer, tablet computer, or the like. However, portable computers are not so limited and may also include other types of portable computers such as cellular telephones, display pagers, radio frequency (RF) devices, infrared (IR) devices, Personal Digital Assistants (PDAs), handheld computers, wearable computers, integrated devices combining one or more of the preceding computers, or the like. As such, computing device 210 may typically range widely in terms of capabilities and features.

Although not shown, computing device 210 may also comprise a network interface that includes circuitry for coupling the computing device to one or more networks, and is constructed for use with one or more communication protocols and technologies including, but not limited to, protocols and technologies that implement any portion of the Open Systems Interconnection model (OSI model), global system for mobile communication (GSM), code division multiple access (CDMA), time division multiple access (TDMA), Long Term Evolution (LTE), user datagram protocol (UDP), transmission control protocol/Internet protocol (TCP/IP), Short Message Service (SMS), Multimedia Messaging Service (MMS), general packet radio service (GPRS), WAP, ultra wide band (UWB), IEEE 802.16 Worldwide Interoperability for Microwave Access (WiMax), Session Initiation Protocol/Real-time Transport Protocol (SIP/RTP), or any of a variety of other wired and wireless communication protocols. In one or more embodiments, the network interface may be a transceiver, or network interface card (NIC).

Additionally, in one or more embodiments (not shown in the figures), computing device 210 may include an one or more embedded logic hardware devices instead of one or more processors, such as, Application Specific Integrated Circuits (ASICs), Field Programmable Gate Arrays (FPGAs), Programmable Array Logic (PALs), or the like, or combination thereof. The one or more embedded logic hardware devices may directly execute its embedded logic to perform actions of the modules discussed above. Also, in one or more embodiments (not shown in the figures), the computing device 210 may include one or more hardware microcontrollers instead of one or more processors. In at least one embodiment, the one or more microcontrollers may directly execute embedded logic to perform actions of the modules and access their own internal memory and their own external Input and Output Interfaces to perform actions, e.g., Systems On Chips (SOCs). Additionally, the storage module shown in FIG. 4 may include one or more of a processor-readable stationary non-transitory storage device or a processor-readable removable non-transitory storage device.

FIGS. 1-4 illustrate the architecture, functionality and operation of possible implementations of systems, methods, processor readable storage media (transitory or non-transitory), and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGS. 1-4. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The method 100 and/or system of the various embodiments can be embodied and/or implemented at least in part as a machine configured to receive a processor-readable media for storing computer-readable instructions. The instructions are preferably executed by processor-executable components preferably integrated with the system and one or more portions of the processor and/or analysis engine. The computer-readable medium can be stored in the cloud and/or on any suitable computer-readable storage media such as RAMs, ROMs, flash memory, EEPROMs, optical devices (CD or DVD), hard drives, floppy drives, or any suitable storage device.

As a person skilled in the art will recognize from the previous detailed description and from the figures and claims, modifications and changes can be made to the preferred embodiments of the invention without departing from the scope of this invention defined in the following claims.

What is claimed as new and desired to be protected by Letters Patent of the United States is:

1. A method for managing treatment for a disorder of a patient, comprising:
employing a set of instructions to generate one or more of an interview dataset and a behavior detection dataset that includes a set of different types of behaviors associated with a patient;
employing the interview dataset to provide one or more tasks for the patient to perform while monitoring the patient for performance of the different types of behaviors in the behavior detection dataset;
transforming the behavior detection dataset into an observation dataset based upon detection of one or more monitored behaviors performed by the patient that corresponds to the behavior detection dataset;
generating a characterization of the disorder of the patient based upon the interview dataset and the observation dataset;
providing one or more goals for one or more therapy regimens for the patient based at least upon the characterization of the patient's disorder;
employing participation by the patient in the one or more therapy regimens over time to generate a set of values for one or more metrics associated with the disorder; and
modifying the one or more therapy regimens based on based on the set of values of the one or more metrics and progress towards meeting the one or more goals.

2. The method of claim 1, further comprising:
providing a task to the patient at a user interface for an application on an electronic device, wherein the task is configured to prompt one or more of the set of different types of behaviors by the patient; and
employing the user interface to automatically detect performance of the one or more different types of behaviors by the patient.

3. The method of claim 1, wherein transforming the behavior detection dataset into the observation dataset further comprises employing video data over time to detect the at least one behavior performed by the patient that corresponds to the set of different types of behaviors.

4. The method of claim 1, wherein transforming the behavior detection dataset into the observation dataset further comprises employing audio data over time to detect the at least one behavior performed by the patient that corresponds to the set of different types of behaviors.

5. The method of claim 1, wherein transforming the behavior detection dataset into the observation dataset further comprises employing one or more sensor devices to detect the at least one behaviors performed by the patient that corresponds to the set of different types of behaviors.

6. The method of claim 1, wherein the provided one or more tasks, further comprise one or more of:
a first task to prompt a neuro-typical behavior by the patent;
a second task to prompt a neuro-atypical behavior by the patient; or
a third task to prompt emotional significance by the patient.

7. The method of claim 1, further comprising:
modifying the characterization of the disorder of the patient based on the set of values of the one or more metrics and progress towards meeting the one or more goals.

8. A system for managing treatment for a disorder of a patient, comprising:
a memory for storing instructions; and
one or more processors that execute the instructions to perform actions, including:
employing a set of instructions to generate one or more of an interview dataset and a behavior detection dataset that includes a set of different types of behaviors associated with a patient;
employing the interview dataset to provide one or more tasks for the patient to perform while monitoring the patient for performance of the different types of behaviors in the behavior detection dataset;
transforming the behavior detection dataset into an observation dataset based upon detection of one or more monitored behaviors performed by the patient that corresponds to the behavior detection dataset;
generating a characterization of the disorder of the patient based upon the interview dataset and the observation dataset;
providing one or more goals for one or more therapy regimens for the patient based at least upon the characterization of the patient's disorder;
employing participation by the patient in the one or more therapy regimens over time to generate a set of values for one or more metrics associated with the disorder; and
modifying the one or more therapy regimens based on based on the set of values of the one or more metrics and progress towards meeting the one or more goals.

9. The system of claim 8, further comprising the actions of:
providing a task to the patient at a user interface for an application on an electronic device, wherein the task is configured to prompt one or more of the set of different types of behaviors by the patient; and
employing the user interface to automatically detect performance of the one or more different types of behaviors by the patient.

10. The system of claim 8, wherein transforming the behavior detection dataset into the observation dataset further comprises employing video data over time to detect the at least one behavior performed by the patient that corresponds to the set of different types of behaviors.

11. The system of claim 8, wherein transforming the behavior detection dataset into the observation dataset further comprises employing audio data over time to detect the at least one behavior performed by the patient that corresponds to the set of different types of behaviors.

12. The system of claim 8, wherein transforming the behavior detection dataset into the observation dataset further comprises employing one or more sensor devices to detect the at least one behaviors performed by the patient that corresponds to the set of different types of behaviors.

13. The system of claim 8, wherein the provided one or more tasks, further comprise one or more of:
a first task to prompt a neuro-typical behavior by the patent;
a second task to prompt a neuro-atypical behavior by the patient; or
a third task to prompt emotional significance by the patient.

14. The system of claim 8, further comprising the actions of:
modifying the characterization of the disorder of the patient based on the set of values of the one or more metrics and progress towards meeting the one or more goals.

15. A processor readable non-transitory storage media that includes instructions for managing treatment for a disorder of a patient, wherein execution of the instructions by one or more processors performs actions, comprising:
employing a set of instructions to generate one or more of an interview dataset and a behavior detection dataset that includes a set of different types of behaviors associated with a patient;
employing the interview dataset to provide one or more tasks for the patient to perform while monitoring the patient for performance of the different types of behaviors in the behavior detection dataset;
transforming the behavior detection dataset into an observation dataset based upon detection of one or more monitored behaviors performed by the patient that corresponds to the behavior detection dataset;
generating a characterization of the disorder of the patient based upon the interview dataset and the observation dataset;
providing one or more goals for one or more therapy regimens for the patient based at least upon the characterization of the patient's disorder;
employing participation by the patient in the one or more therapy regimens over time to generate a set of values for one or more metrics associated with the disorder; and
modifying the one or more therapy regimens based on based on the set of values of the one or more metrics and progress towards meeting the one or more goals.

16. The media of claim 15, further comprising:
providing a task to the patient at a user interface for an application on an electronic device, wherein the task is configured to prompt one or more of the set of different types of behaviors by the patient; and
employing the user interface to automatically detect performance of the one or more different types of behaviors by the patient.

17. The media of claim 15, wherein transforming the behavior detection dataset into the observation dataset further comprises employing video data over time to detect the at least one behavior performed by the patient that corresponds to the set of different types of behaviors.

18. The media of claim 15, wherein transforming the behavior detection dataset into the observation dataset further comprises employing audio data over time to detect the at least one behavior performed by the patient that corresponds to the set of different types of behaviors.

19. The media of claim 15, wherein transforming the behavior detection dataset into the observation dataset further comprises employing one or more sensor devices to detect the at least one behaviors performed by the patient that corresponds to the set of different types of behaviors.

20. The media of claim 15, wherein the provided one or more tasks, further comprise one or more of:
a first task to prompt a neuro-typical behavior by the patent;
a second task to prompt a neuro-atypical behavior by the patient; or
a third task to prompt emotional significance by the patient.
